(19) United States
(12) Patent Application Publication
(10) Pub. No.: US 2006/0173190 A1
(43) Pub. Date: Aug. 3, 2006

(54) Nematicidal thiazoline-containing fluorobutenes

(75) Inventors: Yukiyoshi Watanabe, Tochigi (JP); Jun Mihara, Tochigi (JP); Daiei Yamazaki, Tochigi (JP); Yuichi Otsu, Tochigi (JP); Katsuhiko Shibuya, Tochigi (JP); Eiichi Shimojo, Tochigi (JP)

Correspondence Address: BAYER CROPSCIENCE LP;Patent Department, 100 BAYER ROAD, PITTSBURGH, PA, 15205-9741, US

(73) Assignee: BAYER CROPSCIENCE AG, Monheim (DE)

(21) Appl. No.: 10/560,556

(22) PCT Filed: Jun. 7, 2004
(86) PCT No.: PCT/EP04/06125

§ 371 Date: Feb. 21, 2006

(30) Foreign Application Priority Data
Jun. 19, 2003 (JP) 2003-174758

(51) Int. Cl. A61K 31/426; C07D 277/36

(52) U.S. Cl. 548/182; 514/369

(57) ABSTRACT

The present invention relates to novel thiazoline-containing fluorobutenes of formula (I) 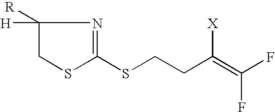 wherein R represents methyl or ethyl, and X represents hydrogen or fluorine, to a process for their preparation and to their use as nematicides.

The present invention relates to novel thiazoline-containing fluorobutenes and their application as a nematicidal agent.

WO 86/07590 A1 describes that certain polyhaloalkene compounds have nematicidal activities. U.S. Pat. No. 3,513,172 and GB 2 293 380 A also describe certain trifluorobutenyl compounds having nematicidal properties. WO 95/04727 describes preparation processes for nematicidal floroalkenylthioheterocyclic derivatives. Finally, WO 95/24403 describes that 4,4-difluoro-butenyl compounds have nematicidal properties.

There have now been found novel thiazoline-containing fluorobutenes of the formula (I) 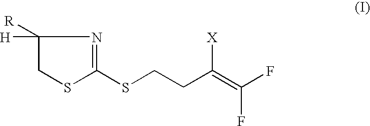
wherein

R represents methyl or ethyl, and

X represents hydrogen or fluorine.

The compounds of the above-mentioned formula (I) can be synthesized, for example, by the following process (a).

Process (a)

Compounds of the formula (II) 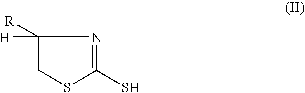
wherein

R has the above-mentioned meaning,

are reacted with compounds of the formula (III) 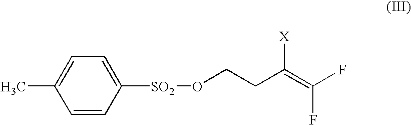
wherein

X has the above-mentioned meaning,

in the presence of inert solvents, and, if appropriate, in the presence of an acid binder.

The compounds of the formula (I), according to the present invention, have strong nematicidal activity and show good compatibility with crops.

According to the present invention, the compounds of the formula (I) surprisingly show very outstanding nematicidal action compared with the compounds described in the aforementioned documents, which are similar to the compounds of the present invention.

Above all, the compounds of the formula (I) are not specifically disclosed in WO 86/07590, although they are conceptually included in the polyhaloalkene compounds described in the above-mentioned WO 86/07590 A1. In the present invention it was found that the compounds of the formula (I) show an outstanding nematicidal activity compared with the compounds disclosed in WO 86/07590.

Preferred substituents or ranges of the radicals present in the formulae given above and below are defined as below:

R preferably represents methyl.

X preferably represents-hydrogen or fluorine.

R particularly preferably represents methyl.

X particularly preferably represents hydrogen.

The compounds of the formula (I) according to the present invention have optical isomers wherein the carbon atom at the 4-position of the thiazoline ring acts as a chiral center. The compounds of the formula (I) according to the present invention also relate to said optical isomers and mixtures of such optical isomers.

Process (a) which can be used to prepare the compounds of the formula (I) can be illustrated by the following reaction scheme. Using, for example, 4-methyl-2-thiazoline-2-thiol and 4,4-difluoro-3-butenyl 4-methylbenezenesulfonate as starting materials, the course of the reaction in the process according to the invention can be illustrated as follows:

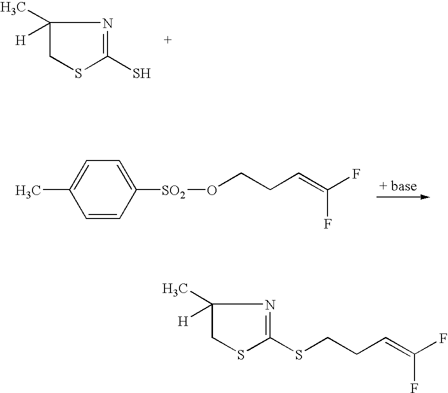

The compounds of the formula (II), used as starting material in the aforementioned process (a), include known compounds which have been described, for example, in U.S. Pat. No. 2,364,398 A. They can be synthesized according to the process described in said U.S. Patent. For example, the process of obtaining 4-methyl-2-thiazoline-2-thiol by reacting 2-amino-1-propanol with carbon disulfide has been described in U.S. Pat. No. 2,364,398 A (see examples).

Specific examples for the compounds of the formula (II) which shall be mentioned are 4-methyl-2-thiazoline-2-thiol, (R)— or (S)-4-methyl-2-thiazoline-2-thiol, 4-ethyl-2-thiazoline-2-thiol, and (R)— or (S)-4-ethyl-2-thiazoline-2-thiol.

The optically isomeric R-modification and S-modification of the above-mentioned compounds can be easily obtained by reacting the respective known optical isomer of 2-amino-1-propanol with carbon disulfide.

The compounds of the formula (I) include known compounds which have been described in WO 95/24403 A1. They can be obtained easily according to the process described in WO 95/24403 A1 as will be shown later in a synthesis reference example.

Specific examples for the compounds of the formula (III) which shall be mentioned are 4,4-difluoro-3-butenyl 4-methylbenzenesulfonate and 3,4,4-trifluoro-3-butenyl 4-methylbenzene-sulfonate.

The process according to the invention for preparing the compounds of the general formula (I) is preferably carried out using an adequate diluent. Suitable diluents for carrying out the process according to the invention are especially inert solvents. These include, in particular, aliphatic, alicyclic and aromatic hydrocarbons, for example, hexane, cyclohexane, petroleum ether, ligroine, benzene, toluene, xylene, etc.; ethers, for example, diethyl ether, methyl ethyl ether, di-isopropyl ether, dibutyl ether, dioxane, tetrahydrofuran, etc.; ketones, for example, acetone, methyl ethyl ketone, methyl isobutyl ketone, etc.; nitriles, for example, acetonitrile, propionitrile, acrylonitrile etc.; acid amides, for example, dimethylformamide, dimethylacetamide, N-methylpyrrolidone, etc.

The process according to the invention for preparing the compounds of the general formula (I) is preferably carried out using an acid binder. Suitable acid binders for carrying out the process according to the invention are, for example, hydroxides, carbonates and alcoholates of alkali metals; tertiary amines, for example, triethylamine, diethylaniline, pyridine, 4-dimethyl-aminopyridine, 1,4-diazabicyclo[2,2,2]octane (DABCO), 1,8-diazabicyclo[5,4,0]undec-7-ene (DBU), etc.

When carrying out the process according to the invention, the reaction temperatures can be varied within a relatively wide range. In general, the process is carried out at temperatures between 0° C. and 180° C., preferably between 20° C. and 120° C.

The process according to the invention is generally carried out under atmospheric pressure. However, it is also possible to carry out the process according to the invention under elevated or reduced pressure—in general between 0.1 bar and 10 bar.

A compound of the formula (I) can be obtained, for example, by reacting 0.7-1.2 moles of a compound of the formula (III) with 1 mole of a compound of the formula (II) in an inert solvent, for example, acetonitrile in the presence of 0.9-1.1 moles of an acid binder, for example, potassium carbonate, under refluxing.

Compounds of the formula (1) wherein X is fluorine can be obtained easily and alternatively by reacting the compounds of the aforementioned formula (II) with 4-bromo-1,1,2-trifluoro-1-butene. 4-Bromo-1,1,2-trifluorobutene is a known compound described in, for example, WO 86/07590 A1. The reaction can be conducted according to the process described in said document.

The compounds of the formula (I) of the present invention show a strong nematicidal activity. They can, therefore, be efficiently used as nematicidal agents, for example, in the field of agriculture and forestry. Remarkably, the compounds of the formula (I) of the present invention are not phytotoxic while at the same time they are effectively controlling harmful nematodes.

The compounds according to the invention can be used, for example, against nematodes such as Pratylenchus spp., Globodera spp., such as Globodera rostochiensis wollenweber, Heterodera spp., such as Heterodera glycines ichinohe, Meloidogyne spp., Aphelenchoides spp., such as Aphelenchoides basseyi christie, Radopholus similis, Ditylenchus dipsaci, Tylenchulus semipenetrans, Longidorus spp., Xiphinema spp., Trichodorus spp., Bursaphelenchus spp., such as Bursaphelenchus xylophilis etc.

The compounds according to the invention are especially useful for combating Pratylenchus spp., Globodera rostochiensis wollenweber, Heterodera glycines ichinohe, Meloidogyne spp., Aphelenchoides basseyi christie, Bursaphelenchus xylophilis.

However, the use of the active compounds according to the invention is in no way restricted to these genera, but also extends in the same manner to other nematodes.

The active compounds of the present invention can be used also in a mixture with other active compounds, for example, insecticides, bactericides, miticides, fungicides, etc. in the form of their commercially useful formulations or in the application forms prepared from such formulations. Insecticides which can be used are, for example, organophosphorous agents, carbamate agents, carboxylate type chemicals, chlorinated hydrocarbon type chemicals, chloronicotinyl type chemicals, insecticidal substances produced by microorganisms, etc.

Further, the active compounds of the present invention can be used also in a mixture with a synergist. Such formulations and application forms can be mentioned as being commercially especially useful. Said synergist must not be active itself, but is a compound that enhances the action of the active compound.

The content of the active compounds of the present invention in a commercially useful formulation or application form can be varied in a wide range. The active-compound content of the use forms prepared from the commercial formulations can vary within wide limits. The active-compound concentration of the use forms can be from 0.0000001 to 100% by weight of active compound, preferably between 0.0001 and 1% by weight.

Examples of advantageous mixing components are, for-example, the following:

Fungicides

aldimorph, ampropylfos, ampropylfos potassium, andoprim, anilazine, azaconazole, azoxystrobin, benalaxyl, benodanil, benomyl, benzamacril, benzamacril-isobutyl, bialaphos, binapacryl, biphenyl, bitertanol, blasticidin-S, bromuconazole, bupirimate, buthiobate, calcium polysulphide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram, debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon, ediphenphos, epoxiconazole, etaconazole, ethirimol, etridiazole, famoxadon, fenapanil, fenarimol, fenbuconazole, fenfuram, fenitropan, fenpiclonil, fenpropidin, fenpropimorph, fentin acetate, fentin hydroxide, ferbam, ferimzone, fluazinam, flumetover, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, fuirmecyclox, guazatine, hexachlorobenzene, hexaconazole, hymexazole, imazalil, imibenconazole, iminoctadine, iminoctadine albesilate, iminoctadine triacetate, iodocarb, ipconazole, iprobenfos (IBP), iprodione, irumamycin, isoprothiolane, isovaledione, kasugamycin, kresoxim-methyl, copper preparations, such as: copper hydroxide, copper naphthenate, copper oxychloride, copper sulphate, copper oxide, oxine-copper and Bordeaux mixture, mancopper, mancozeb, maneb, meferimzone, mepanipyrim, mepronil, metalaxyl, metconazole, methasulfocarb, metbfuroxam, metiram, metomeclam, metsulfovax, mildiomycin, myclobutanil, myclozolin, nickel dimethyldithiocarbamate, nitrothal-isopropyl, nuarimol, ofurace, oxadixyl, oxamocarb, oxolinic acid, oxycarboxim, oxyfenthiin, paclobutrazole, pefurazoate, penconazole, pencycuron, phosdiphen, pimaricin, piperalin, polyoxin, polyoxorim, probenazole, prochloraz, procymidone, propamocarb, propanosine-sodiurn, propiconazole, propineb, pyrazophos, pyrifenox, pyrimethanil, pyroquilon, pyroxyfur, quinconazole, quintozene (PCNB), sulphur and sulphur preparations, tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, triflumizole, triforine, triticonazole, uniconazole, validamycin A, vinclozolin, viniconazole, zarilamide, zineb, ziram and also Dagger G, OK-8705, OK-8801, α-(1′,1-dimethylethyl)-β-(2-phenoxyethyl)-1H-1,2,4-triazole-1-ethanol, α-(2,4-dichlorophenyl)-β-fluoro-b-propyl-1H-1,2,4-triazole-1-ethanol, α-(2,4-dichlorophenyl)-β-methoxy-a-methyl-1H-1,2,4-triazole-1-ethanol, α-(5-methyl-1,3-dioxan-5-yl)-β-[[4-(trifluoromethyl)-phenyl]-meth-ylene]-1H-1,2,4-triazole-1-ethanol, (5RS,6RS)-6-hydroxy-2,2,7,7-tetramethyl-5-(1H-1,2,4-triazol-1-yl)-3-octanone, (E)-a-(methoxyimino)-N-methyl-2-phenoxy-phenylacetamide, isopropyl 1-{2-methyl-1-[[[1-(4-methylphenyl)-ethyl]-amino]-carbonyl]-propyl)-carbamate, 1-(2,4-dichloro-phenyl)-2-(1H-1,2,4-triazol-1-yl)-ethanone O-(phenylmethyl)oxime, 1-(2-methyl-1-naphthalenyl)-1H-pyrrol-2,5-dione, 1-(3,5-dichlorophenyl)-3-(2-propenyl)-2,5-pyrrolidinedione, 1-[(diiodomethyl)-sulphonyl]-4-methyl-benzene, 1-[[2-(2,4-dichlorophenyl)-1,3-dioxolan-2-yl]-methyl]-1H-imidazole, 1-[[2-(4-chlorophenyl)-3-phenyloxiranyl]-methyl]-1H-1,2,4-triazole, 1-[1-[2-[(2,4-dichlorophenyl)-methoxy]-phenyl]-ethenyl]-1H-imidazole, 1-methyl-5-nonyl-2-(phenyl-methyl)-3-pyrrolidinole, 2′,6′-dibromo-2-methyl-4′-trifluoromethoxy-4′-trifluoro-methyl-1,3-thiazole-5-carboxanilide, 2,2-dichloro-N-[1-(4-chlorophenyl)-ethyl]-1-ethyl-3-methyl-cyclopro-panecarboxamide, 2,6-dichloro-5-(methylthio)-4-pyrimidinyl thiocyanate, 2,6-dichloro-N-(4-trifluoromethylbenzyl)-benzamide, 2,6-dichloro-N-[[4-(trifluoromethyl)-phenyl]-methyl]-benz-amide, 2-(2,3,3-triiodo-2-propenyl)-2H-tetrazole, 2-[(1-methylethyl)-sulphonyl]-5-(trichloro-methyl)-1,3,4-thiadiazole, 2-[[6-deoxy-4-O-(4-O-methyl-β-D-glycopyranosyl)-a-D-gluco-pyranosyl]-amino]-4-methoxy-1H-pyrrolo[2,3-d]pyrimidine-5-carbonitrile, 2-aminobutane, 2-bromo-2-(bromomethyl)-pentanedinitrile, 2-chloro-N-(2,3-dihydro-1,1,3-trimethyl-1H-inden-4-yl)-3-pyridinecarboxamide, 2-chloro-N-(2,6-dimethylphenyl)-N-(isothiocyanatomethyl)-acet-amide, 2-phenylphenol (OPP), 3,4-dichloro-1-[4-(difluoromethoxy)-phenyl]-1H-pyrrol-2,5-dione, 3,5-dichloro-N-[cyano-[(1-methyl-2-propynyl)-oxy]-methyl]-benzamide, 3-(1,1-dimethylpropyl-1-oxo-1H-indene-2-carbonitrile, 3-[2-(4-chlorophenyl)-5-ethoxy-3-isoxazolidinyl]-pyridine, 4-chloro-2-cyano-N,N-dimethyl-5-(4-methylphenyl)-1H-imidazole-1-sulphonamide, 4-methyl-tetra-zolo[1,5-a]quinazolin-5(4H)-one, 8-(1,1-dimethylethyl)-N-ethyl-N-propyl-1,4-dioxa-spiro[4.5]decane-2-methanamine, 8-hydroxyquinoline sulphate, 9H-xanthene-2-[(phenylamino)-carbonyl]-9-carboxylic hydrazide, bis-(1-methylethyl) 3-methyl-4-[(3-methylbenzoyl)-oxy]-2,5-thiophenedicarboxylate, cis-1-(4-chlorophenyl)-2-(1H-1,2,4-triazol-1-yl)-cycloheptanol, cis-4-[3-[4-(1,1-dimethylpropyl)-phenyl-2-methylpropyl]-2,6-dimethyl-morpholine hydrochloride, ethyl [(4-chlorophenyl)-azo]-cyanoacetate, potassium hydrogen carbonate, methanetetrathiol sodium salt, methyl 1-(2,3-dihydro-2,2-dimethyl-1H-inden-1-yl)-1H-imidazole-5-carboxylate, methyl N-(2,6-dimethylphenyl)-N-(5-isoxazolylcarbonyl)-DL-alaninate, methyl N-(chloroacetyl)-N-(2,6-dimethylphenyl)-DL-alaninate, N-(2,3-dichloro-4-hydroxyphenyl)-1-methyl-cyclohexanecarb-oxamide, N-(2,6-dimethylphenyl)-2-methoxy-N-(tetrahydro-2-oxo-3-furanyl)-acetamide, N-(2,6-dimethylphenyl)-2-methoxy-N-(tetrahydro-2-oxo-3-thienyl)-acetamide, N-(2-chloro-4-nitro-phenyl)-4-methyl-3-nitro-benzenesulphonamide, N-(4-cyclohexylphenyl)-1,4,5,6-tetrahydro-2-pyrimidineamine, N-(4-hexylphenyl)-1,4,5,6-tetrahydro-2-pyrimidineamine, N-(5-chloro-2-meth-ylphenyl)-2-methoxy-N-(2-oxo-3-oxazolidinyl)-acetamide, N-(6-methoxy)-3-pyridinyl)-cyclo-propanecarboxamide, N-[2,2,2-trichloro-1-[(chloroacetyl)-amino]-ethyl]-benzamide, N-[3-chloro-4,5-bis(2-propinyloxy)-phenyl]-N′-methoxy-methanimidamide, N-formyl-N-hydroxy-DL-alanine-sodium salt, O,O-diethyl[2-(dipropylamino)-2-oxoethyl]-ethylphosphoramidothioate, O-methyl S-phenyl phenylpropylphosphoramidothioate, S-methyl 1,2,3-benzothiadiazole-7-carbothioate, and spiro[2H]-1-benzopyran-2,1′(3′H)-isobenzofuran]-3′-one.

Bactericides

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Insecticides/Acaricide/Nematicides

abamectin, acephate, acetamiprid, acrinathrin, alanycarb, aldicarb, aldoxycarb, alpha-cypermethrin, alphamethrin, amitraz, avermectin, AZ 60541, azadirachtin, azamethiphos, azinphos A, azinphos M, azocyclotin, Bacillus popilliae, Bacillus sphaericus, Bacillus subtilis, Bacillus thuringiensis, baculoviruses, Beauveria bassiana, Beauveria tenella, benclothiaz, bendiocarb, benfuracarb, bensultap, benzoximate, betacyfluthrin, bifenazate, bifenthrin, bioethanomethrin, biopermethrin, BPMC, bromophos A, bufencarb, buprofezin, butathiofos, butocarboxim, butylpyridaben, cadusafos, carbaryl, carbofuran, carbophenothion, carbosulfan, cartap, chloethocarb, chlorethoxyfos, chlorfenapyr, chlorfenvinphos, chlorfluazuron, chlormephos, chlorpyrifos, chlorpyrifos M, chlovaporthrin, cis-resmethrin, cispermethrin, clocythrin, cloethocarb, clofentezine, cyanophos, cycloprene, cycloprothrin, cyfluthrin, cyhalothrin, cyhexatin, cypermethrin, cyromazine, deltamethrin, demeton M, demeton S, demeton-S-methyl, diafenthiuron, diazinon, dichlorvos, diflubenzuron, dimefluthrin, dimethoat, dimethylvinphos, diofenolan, disulfoton, docusat-sodium, dofenapyn, eflusilanate, emamectin, empenthrin, endosulfan, Entomopfthora spp., esfenvalerate, ethiofencarb, ethion, ethoprophos, etofenprox, etoxazole, etrimfos, fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluazinam, fluazuron, flubrocythrinate, flucycloxuron, flucythrinate, flufenoxuron, flutenzine, fluvalinate, fonophos, fosmethilan, fosthiazate, fubfenprox, furathiocarb, gamma-cyhalothrin, granulosis viruses, halofenozide, HCH, heptenophos, hexaflumuron, hexythiazox, hydroprene, imidacloprid, isazofos, isofenphos, isoxathion, ivermectin, nuclear polyhedrosis viruses, lambda-cyhalothrin, lufenuron, malathion, mecarbam, metaldehyde, methamidophos, Metharhizium anisopliae, Metharhizium flavoviride, methidathion, methiocarb, methomyl, methoxyfenozide, metofluthrin, metolcarb, metoxadiazone, mevinphos, milbemectin, monocrotophos, naled, nitenpyram, nithiazine, novaluron, omethoat, oxamyl, oxydemethon M, Paecilomyces fumosoroseus, parathion A, parathion M, permethrin, phenthoat, phorat, phosalone, phosmet, phosphamidon, phoxim, pirimicarb, pirimiphos A, pirimiphos M, profenofos, potassium oleate, prallethrin, profluthrin, promecarb, propoxur, prothiofos, prothoat, pymetrozine, pyraclofos, pyresmethrin, pyrethrum, pyridaben, pyridathion, pyrimidifen, pyriproxyfen, quinalphos, ribavirin, salithion, sebufos, silafluofen, spinosad, sulfotep, sulprofos, tau-fluvalinate, tebufenozide, tebufenpyrad, tebupirimiphos, teflubenzuron, tefluthrin, temephos, temivinphos, terbufos, tetrachlorvinphos, theta-cypermethrin, thiamethoxam, thiapronil, thiatriphos, thiocyclam hydrogen oxalate, thiodicarb, thiofanox, thuringiensin, tralocythrin, tralomethrin, triarathene, triazamate, triazophos, triazuron, trichlophenidine, trichlorfon, Trichoderma atroviride, triflumuron, trimethacarb, vamidothion, vaniliprole, Verticillium lecanii, YI 5302, zeta-cypermethrin, zolaprofos, (1R-cis)-[5-(phenylmethyl)-3-furanyl]-methyl 3-[(dihydro-2-oxo-3(2H)-furanylidene)-methyl]-2,2-dimethylcyclo-propanecarboxylate, (3-phenoxyphenyl)-methyl 2,2,3,3-tetramethylcyclopropanecarboxylate, 1-[(2-chloro-5-thiazolyl)methyl]tetrahydro-3,5-dimethyl-N-nitro-1,3,5-triazine-2(1H)-imine, 2-(2-chloro-6-fluorophenyl)-4-[4-(1,1-dimethylethyl)phenyl]-4,5-dihydro-oxazole, 2-(acetlyoxy)-3-dodecyl-1,4-naphthalenedione, 2-chloro-N-[[[4-(1-phenylethoxy)-phenyl]-amino]-carbonyl]-benzamide, 2-chloro-N-[[[4-(2,2-dichloro-1,1-difluoroethoxy)-phenyl]-amino]-carbonyl]-benzamide, 3-methylphenyl propylcarbamate, 4-[4-(4-ethoxyphenyl)-4-methylpentyl]-1-fluoro-2-phenoxy-benzene, 4-chloro-2-(1,1-dimethylethyl)-5-[[2-(2,6-dimethyl-4-phenoxyphenoxy)ethyl]thio]-3(2H)-pyridazinone, 4-chloro-2-(2-chloro-2-methylpropyl)-5-[(6-iodo-3-pyridinyl)-methoxy]-3(2H)-pyridazinone, 4-chloro-5-[(6-chloro-3-pyridinyl)methoxy]-2-(3,4-dichloro-phenyl)-3(2H)-pyridazinone, Bacillus thuringiensis strain EG-2348, [2-benzoyl-1-(1,1-dimethylethyl)-hydrazinobenzoic acid, 2,2-dimethyl-3-(2,4-dichlorophenyl)-2-oxo-1-oxa-spiro[4.5]dec-3-en-4-yl butanoate, [3-[(6-chloro-3-pyridinyl)methyl]-2-thiazolidinylidene]-cyanamide, dihydro-2-(nitromethylene)-2H-1,3-thiazine-3(4H)-carboxaldehyde, ethyl [2-[[1,6-dihydro-6-oxo-1-(phenylmethyl)-4-pyridazinyl]oxy]ethyl]-carbamate, N-(3,4,4-trifluoro-1-oxo-3-butenyl)-glycine, N-(4-chlorophenyl)-3-[4-(difluoromethoxy)phenyl]-4,5-dihydro-4-phenyl-1H-pyrazole-1-carboxamide, N-[(2-chloro-5-thiazolyl)methyl]-N′-methyl-N″-nitro-guanidine, N-methyl-N′-(1-methyl-2-propenyl)-1,2-hydrazinedicarbothioamide, N-methyl-N′-2-propenyl-1,2-hydrazinedicarbothioamide, O,O-diethyl [2-(dipropylamino)-2-oxoethyl]-ethylphosphoroamido-thioate.

A mixture with other known active compounds, such as herbicides, or with fertilizers and growth regulators is also possible.

The active compounds of the present invention can be converted into customary formulations such as solutions, emulsions, wettable powders, water-dispersible granules, suspensions, powders, foaming agents, pastes, granules, active compound-impregnated natural and synthetic substances, microcapsules, fumigants etc.

These formulations can be prepared according to per se known methods, for example, by mixing the active compounds with extenders, namely liquid, liquefied gas or solid diluents or carriers, and optionally with surface-active agents, namely emulsifiers and/or dispersants and/or foam-forming agents. If the extender used is water, it is also possible to use, for example, organic solvents as auxiliary solvents. Suitable liquid solvents are essentially: aromatics, such as xylene, toluene, or alkylnaphthalenes, chlorinated aromatics and chlorinated aliphatic hydrocarbons, such as chloro-benzene, chloroethylenes or methylene chloride, aliphatic hydrocarbons, such as cyclohexane or paraffins, for example mineral oil fractions, mineral or vegetable oil, alcohols, such as butanol or glycol, and also their ethers and esters, ketones, such as acetone, methyl ethyl ketone, methyl isobutyl ketone or cyclohexanone, strongly polar solvents, such as dimethylformamide and dimethyl sulphoxide, and also water.

Liquid diluents or carriers can be, for example, aromatic hydrocarbons (for example, xylene, toluene, alkylnaphthalene etc.), chlorinated aromatic or chlorinated aliphatic hydrocarbons (for example, chlorobenzenes, ethylene chlorides, methylene chloride etc.), aliphatic hydrocarbons (for example, cyclohexane etc. or paraffins, such as, mineral oil fractions etc.), alcohols (for example, butanol, glycols and their ethers, esters etc.), ketones (for example, acetone, methyl ethyl ketone, methyl isobutyl ketone, cyclohexanone etc.), strongly polar solvents (for example, dimethylformamide, dimethyl sulfoxide etc.), water etc.

Liquefied gas diluents or carriers are liquefied substances which are gases at normal temperature and pressure. Liquefied gas diluents can be, for example, aerosol propellants such as butane, propane, nitrogen gas, carbon dioxide, halogenated hydrocarbons, etc.

Solid diluents can be, for example, ground natural minerals (for example, kaolin, clay, talc, chalk, quartz, attapulgite, montmorillonite, diatomaceous earth etc.), ground synthetic minerals (for example, highly dispersed silicic acid, alumina, silicates etc.) etc.

Solid carriers for granules can be, for example, crushed and fractionated rocks (for example, calcite, marble, pumice, sepiolite, dolomite etc.) synthetic granules of inorganic and organic meals, particles of organic materials (for example, saw dust, coconut shells, maize cobs, tobacco stalks etc.) etc.

Emulsifiers and/or foam-forming agents can be, for example, nonionic and anionic emulsifiers, for example, polyoxyethylene fatty acid esters, polyoxyethylene fatty acid alcohol ethers, such as, alkylaryl polyglycol ethers, alkylsulfonates, alkylsulfates, arylsulfonates etc., albumin hydrolysis products etc.

Dispersants include, for example, lignin sulfite waste liquor, methyl cellulose etc.

Tackifiers can also be used in formulations (powders, granules, emulsifiable concentrates). As usable tackifiers there can be mentioned, for example, carboxymethyl cellulose, natural and synthetic polymers (for example, gum Arabic, polyvinyl alcohol, polyvinyl acetate etc.).

Colorants can also be used. Colorants can be, for example, inorganic pigments (for example, iron oxide, titanium oxide, Prussian Blue etc,), organic dyestuffs such as alizarin dyestuffs, azo dyestuffs or metal phthalocyanine dyestuffs, and further traces nutrients such as salts of metals such as iron, manganese, boron, copper, cobalt, molybdenum, zinc etc.

Said formulations can contain the aforementioned active components in a range of generally 0.1-95% by weight, preferably 0.5-90% by weight.

The preparation and possible application forms of the compounds of the present invention will be described more specifically by the following examples. The present invention, however, should not be restricted to them in any way. “Parts” means “parts by weight” unless otherwise specified.

### EXAMPLES

### Synthesis Example 1

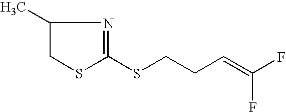

1 g (7.51 mmol) of 4-methyl-2-thiazoline-2-thiol, 1.24 g (9.01 mmol) of potassium carbonate and 1.77 g (6.76 mmol) of 4,4-difluoro-3-butenyl 4-methylbenzenesulfonate were suspended in 30 ml of acetonitrile and the suspension was refluxed for 4 hours. After removing the precipitates, the filtrate was concentrated under reduced pressure and the residue was purified by column chromatography (n-hexane: ethyl acetate=9:1) to obtain 1.23 g of 2-(4′,4′-difluoro-3′-butenylthio)-4-methyl-2-thiazoline. n_{D}²⁰=1.5120, yield 73%.

The compounds of the formula (I), according to the present invention, which can be obtained in the same way as described in the above example 1, are shown in the following Table 1, together with the compound of Synthesis Example 1.

TABLE 1

| | |
|---|---|
| | (I) |
| 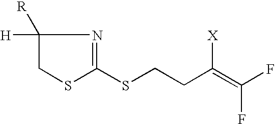 | |

| Compound | | | Stereochemical | |
|---|---|---|---|---|
| No. | R | X | character | Property n_{D}²⁰ |
| 1 | CH₃ | H | Racemic | 1.5120 |
| 2 | CH₃ | H | R— | 1.5090 |
| 3 | CH₃ | H | S— | 1.5230 |
| 4 | C₂H₅ | H | Racemic | 1.5095 |
| 5 | CH₃ | F | Racemic | 1.4945 |
| 6 | CH₃ | F | S— | 1.4905 |
| 7 | CH₃ | F | R— | 1.5020 |
| 8 | C₂H₅ | H | R— | 1.5075 |
| 9 | C₂H₅ | H | S— | 1.5092 |
| 10 | C₂H₅ | F | Racemic | |
| 11 | C₂H₅ | F | R— | |
| 12 | C₂H₅ | F | S— | |

### Synthesis Example 2 (Alternative Process)

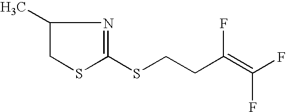

0.5 g (3.75 mmol) of 4-methyl-2-thiazoline-2-thiol, 0.62 g (4.50 mmol) of potassium carbonate and 0.64 g (3.38 mmol) of 4-bromo-1,1,2-trifluoro-1-butene were suspended in 25 ml of acetonitrile and the suspension was refluxed for 4 hours. After filtering off the precipitates, the filtrate was concentrated under reduced pressure and the residue was purified by column chromatography (n-hexane: ethyl acetate=9:1) to obtain 0.71 g of 2-(3′,4′,4′-trifluoro-3′-butenylthio)-4-methyl-2-thiazoline. n_{D}²⁰=1.4945, yield 78%.

### Synthesis Example 3 (Starting Material)

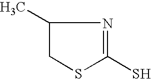

8 g (200 mmol) of sodium hydroxide was dissolved in 14.4 g of water and 7.51 g (100 mmol) of 2-amino-1-propanol was added thereto. Further, 21.32 g (280 mmol) of carbon disulfide was added thereto under ice cooling and the mixture was refluxed for 7 hours. After cooling, the mixture was acidified with concentrated hydrochloric acid and extracted with dichloromethane. The dichloro-methane layer was dried with anhydrous magnesium sulfate and the solvent was distilled off under reduced pressure. The residue was purified by column chromatography (n-hexane: ethyl acetate 3:2) to obtain 4.68 g of 4methyl-2-thiazoline-2-thiol. mp.: 98-100° C., yield: 35%.

### Synthesis Example 4 (Starting Material)

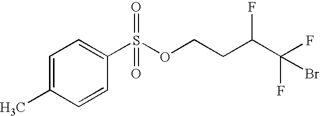

24.92 g (89.30 mmol) of silver p-toluenesulfonate and 24.10 g (89.30 mmol) of 1,4-dibromo-1,1,2-trifluorobutane were suspended in 200 ml of acetonitrile and the suspension was refluxed for 7 hours. After cooling, the precipitates were removed. The filtrate was concentrated under reduced pressure and the obtained residue was mixed with water and then extracted with ethyl acetate. The ethyl acetate layer was dried with anhydrous sodium sulfate and the solvent was distilled off under reduced pressure. The residue was treated by column chromatography (n-hexane: ethyl acetate=6:1) to obtain 27.77 g of 4-bromo-3,4,4-trifluorobutyl 4-methyl-benzenesulfonate. n_{D}²⁰: 1.4888, yield: 86%.

### Synthesis Example 5 (Starting Material)

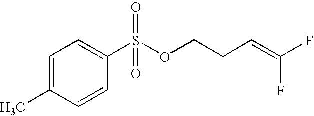

27.77 g (76.89 mmol) of 4-bromo-3,4,4-trifluorobutyl 4-methylbenzenesulfonate, 55.3 g (845.76 mmol) of zinc and a catalytic amount of iodine were suspended in 150 ml of methanol and the suspension was refluxed for 2.5 hours. After cooling, the precipitates were removed. The filtrate was concentrated under reduced pressure and the obtained residue was mixed with ethyl acetate and washed with 10% hydrochloric acid. The ethyl acetate layer was dried with anhydrous sodium sulfate and the solvent was distilled off under reduced pressure. The residue was purified by column chromatography (n-hexane: ethyl acetate=6:1) to obtain 15.99 g of 4,4-difluoro-3-butenyl 4-methylbenzenesulfonate. n_{D}²⁰=1.4885, yield 79%.

### Test Example 1

### Test Against Meloidogyne spp. (Soil Pot Test)

Preparation of Test Agent

1 Part of the active compound is impregnated to 99 parts of pumice to make fine granules.

Test Method

The test agent prepared as mentioned above was added to the soil contaminated by Meloidogyne incognita so that the chemical concentration would be 10 ppm. The soil and the test agent were homogeneously mixed by stirring and a pot (1/5000 are) was filled with the soil. About 20 seeds of tomato (variety: Kurihara) were sown per pot. After cultivation in a greenhouse for 4 weeks, they were carefully pulled out not to damage the roots and the root knot index and the controlling effect were determined as follows.

Degree of damage 0: No knots were formed (Complete control)
1: A few knots were formed.
2: Knots were formed to a medium extent.
3: Knots were formed to an intense extent.
4: Knots were formed to the most intense extent

$$Root knot index = \frac{\begin{matrix}( which corresponds to non \text{-} treatment ) \cdot \\ Σ ( degree of damage \times number of individuals )\end{matrix}}{Total number of tested individuals \times 4} ⨯ 100$$

The controlling effect of the compounds tested can then be evaluated according to the following equation:

$$Controlling effect [ \% ] = \frac{\begin{matrix}( Root knot index at non \text{-} treated area ) - \\ ( Root knot index at treated area )\end{matrix}}{Root knot index at non \text{-} treated area} \times 100$$

In the test described, the following compounds showed more than 90% controlling effect at an effective concentration of 10 ppm: No. 1, 2, 3, 4 and 5.

### Formulation Example 1 (Granules)

To a mixture of 10 parts of a compound according to the invention (No. 1), 30 parts of bentonite (montmorillonite), 58 parts of talc and 2 parts of ligninsulfonate salt, 25 parts of water were added, well kneaded, made into granules of 10-40 mesh by an extrusion granulator and dried at 40-50° C. to obtain granules.

### Formulation Example 2 (Granules)

95 Parts of clay mineral particles having particle diameter distribution of 0.2-2 mm are put in a rotary mixer. While rotating it, 5 parts of a compound according to the invention (No. 1) are sprayed together with a liquid diluent, wetted uniformly and dried at 40-50° C. to obtain granules.

### Formulation Example 3 (Emulsifiable Concentrate)

30 Parts of a compound according to the invention (No. 2), 55 parts of xylene, 8 parts of polyoxyethylene alkyl phenyl ether and 7 parts of calcium alkylbenzenesulfonate are mixed and stirred to obtain an emulsifiable concentrate.

### Formulation Example 4 (Wettable Powder)

15 Parts of a compound according to the invention (No. 2), 80 parts of a mixture of white carbon (hydrous amorphous silicon oxide fine powders) and powder clay (1:5), 2 parts of sodium alkylbenzenesulfonate and 3 parts of sodium alkylnaphthalenesulfonate-formalin-condensate are crushed and mixed to make a wettable powder.

What is claimed is:

1-8. (canceled)

9. A compound of formula (I) 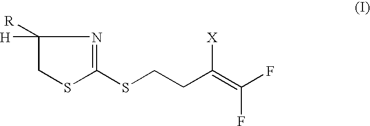
wherein
R represents methyl or ethyl, and
X represents hydrogen or fluorine.

10. A compound of formula (I) according to claim 9 wherein
R represents methyl, and
X represents hydrogen or fluorine.

11. A compound of formula (I) according to claim 9 wherein
R represents methyl, and
X represents hydrogen.

12. A process for preparing compounds of formula (I) according to claim 9 comprising reacting a compound of formula (II) 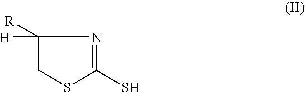
wherein R represents methyl or ethyl, with a compound of formula (III) 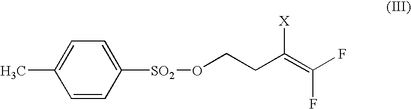
wherein X represents hydrogen or fluorine,
in the presence of inert solvents and optionally in the presence of an acid binder.

13. A nematicidal composition comprising one or more compounds of formula (I) according to claim 9 and one or more extenders and/or surface active agents.

14. A method of combating nematodes comprising allowing an effective amount of a compound of formula (I) according to claim 9 to act on nematodes and/or their environment.

15. A process for preparing a nematicidal composition comprising mixing one or more compounds of formula (I) according to claim 9 with one or more extenders and/or surface active agents and/or other adjuvants.
